(19) United States
(12) Patent Application Publication
(10) Pub. No.: US 2014/0309497 A1
(43) Pub. Date: Oct. 16, 2014

(54) TISSUE PROTECTION/RETRACTION SYSTEM

(71) Applicants: CLIFFORD T. SOLOMON, SEVERNA PARK, MD (US); THEODORE C. SOLOMON, HAMPSTEAD, MD (US)

(72) Inventors: CLIFFORD T. SOLOMON, SEVERNA PARK, MD (US); THEODORE C. SOLOMON, HAMPSTEAD, MD (US)

(21) Appl. No.: 13/863,290

(22) Filed: Apr. 15, 2013

(51) Int. Cl. A61B 17/02; A61B 19/00

(52) U.S. Cl. CPC A61B 17/02; A61B 19/46; A61B 19/54; A61B 2017/00889
USPC 600/202; 600/203

(57) ABSTRACT

The protective tissue cover includes a sleeve configured to slide onto the working portion of a surgical retractor. At least the contact side of the protective tissue cover is constructed from smooth antimicrobial material that minimizes contact irritation with tissue. Alternative embodiments include various multiple-ply constructions that incorporate an antimicrobial layer, an absorbent layer, and other medical layers to assist in surgical procedures.

### BACKGROUND OF THE INVENTION

1. Field of the Invention

The present invention relates to medical devices, and particularly to a protective tissue cover that provides gentle, sterile, and non-aggravating contact with tissue during surgery.

2. Description of the Related Art

In many respects, surgical procedures are very delicate processes. Much care and attention must be maintained not just with the procedure itself but also with the environmental conditions. Any invasive procedure runs the risk of potential contamination, infection, and inflammation as well as the occasional unforeseen complication to the patient. In modern medicine, many guidelines have been developed to minimize such dangers, such as protective apparel, specialized bandages and other surgical equipment, meticulous monitoring of tools and equipment used during surgery, and scrubbing or cleansing procedures for the surgeon, support staff, instruments and the surgical area.

One aspect of surgery that is of much concern is the handling of tissue during surgical procedures. Most conventional surgical procedures utilize surgical drapes around the incision site, providing a sterile barrier to prevent or reduce contamination. Special bandages or towelettes, such as cottonoids, are also used as drapes and sponges. These implements, while sterile, can cause trauma and irritation to the underlying tissue in and around the incision site, especially the delicate tissue during in various surgeries including brain surgery. In some instances, there have been cases of cotton residue left in the surgical site, which is a potential hazardous complication for patient recovery. The residue can result in focal inflammation and/or infection.

Besides the above implements, surgeons routinely use specialized tools, such as retractors, during the surgical procedure as a means of gaining access to critical anatomical sites and to keep surrounding tissue away from the target area. Retractor designs range from simple to complex, and retractors are typically constructed from surgical grade materials. Any contact of the retractors against exposed tissue, usually prolonged in most procedures where surrounding tissue must be isolated, can abrade or irritate the underlying tissue, causing potential inflammation leading to contamination and/or infection. Due to the complex biology of individuals, some patients are more sensitive and susceptible to these types of complications.

In light of the above, it would be a benefit in the art of medical device to provide some means of minimizing these types of complications. Thus, a protective tissue cover solving the aforementioned problems is desired.

### SUMMARY OF THE INVENTION

The protective tissue cover includes a sleeve configured to slide onto the working portion of a surgical retractor. At least the contact side of the protective tissue cover is constructed from smooth antimicrobial material that minimizes contact irritation with tissue. Alternative embodiments include various multiple-ply constructions that incorporate an antimicrobial layer, an absorbent layer, and other medical layers to assist in surgical procedures.

These and other features of the present invention will become readily apparent upon further review of the following specification and drawings.

### BRIEF DESCRIPTION OF THE DRAWINGS

FIG. 1 is an environmental, perspective view of a protective tissue cover according to the present invention.

FIG. 2 is a perspective view of an alternative protective tissue cover according to the present invention in the form of discrete sheets.

FIG. 3 is a sectional view of another alternative protective tissue cover according to the present invention.

FIG. 4 is a sectional view of yet another alternative protective tissue cover according to the present invention with features for positive suctioning of fluids.

Similar reference characters denote corresponding features consistently throughout the attached drawings.

### DETAILED DESCRIPTION OF THE PREFERRED EMBODIMENTS

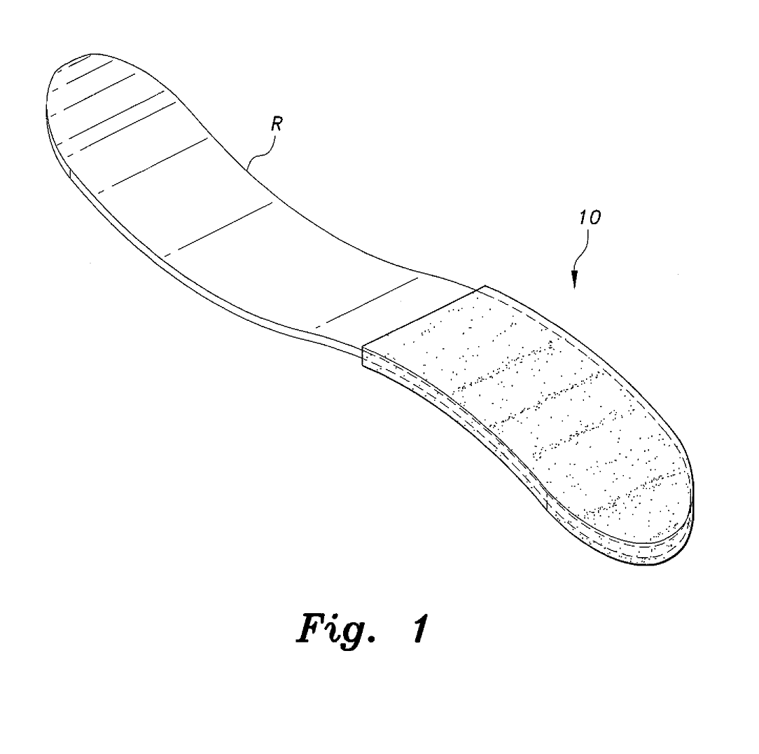

The protective tissue cover, a first embodiment of which is generally referred to by the reference number 10, provides a gentle, minimal irritant, sterile and/or antimicrobial, bactericidal layer between exposed tissue and environmental intrusions. As best seen in FIG. 1, the protective tissue cover 10 is constructed as a sleeve configured to slide onto the working portion of a retractor R. In the drawing, the protective tissue cover 10 is shown attached to a curved ribbon retractor, but it is to be understood that the protective tissue cover 10 can be constructed to fit a variety of configurations of retractors and the like. As an alternative, the protective tissue cover 10 can be used alone to function as a retractor.

The protective tissue cover 10 is preferably made from a smooth, resilient, polymeric material (medical grade) having sterile, antimicrobial and/or antiallergenic properties. The smoothness offers near frictionless contact with tissue through the interaction of natural biological fluids and/or solutions used in surgery between the material and tissue. This minimizes much of the potential abrading and irritation therewith, especially with tissue contact during surgery and the process of removing the retractor. It is to be understood that the benefits herein apply to any tissue that may be in contact with the protective tissue cover 10. Obviously, a sterile environment during surgery must be maintained at all times, and antimicrobial properties assure that this can be accomplished with minimal risk to the patient. As mentioned previously, some patients may exhibit hypersensitivity to certain materials or chemicals, and antiallergenic properties insure that the protective tissue cover 10 can be utilized for as wide a spectrum of patients as possible.

One example of an antimicrobial and antiallergenic material is Ioban®, manufactured by 3M, which is primarily a surgical drape impregnated with iodine. This particular material has been shown to be an effective antimicrobial barrier during surgical procedures. Other types of materials include, but are not limited thereto, medical grade elastomeric material, such as Silastic®, manufactured by Dow Corning, and other silicone elastomers and the like, that are impregnated or synthesized with chlorhexadine, collagen, vitamin D, quaternary amines, halamine, silver, combinations and the like. It is to be understood that some of these chemicals or compounds may exhibit toxicity at high levels of concentration. Thus, the composition must be made with due consideration for minimizing risks of toxicity. Another example includes materials such as Telfa®, a dressing produced by Kendall Company, and the like. Telfa® exhibits anti-adhering properties while providing absorption of fluids.

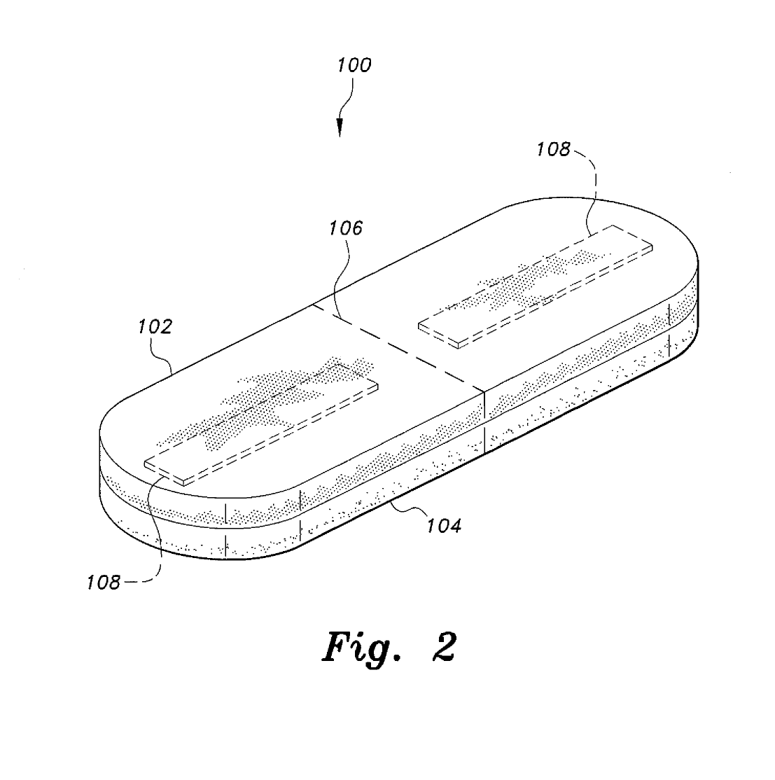

An alternative protective tissue cover 100 is shown in FIG. 2. In this embodiment, the protective tissue cover 100 is constructed in the form of discrete or endless webs or sheets for use as a protective layer for the underlying tissue. As shown, the protective tissue cover 100 has multiple layers of material, including a first or upper layer 102 and a second or lower layer 104. The upper layer 102 is constructed from the same sterile and/or antimicrobial material mentioned above, while the lower layer 104 is preferably constructed from absorbent material, such as Cottonoid® (cottonoid is a trademark of Johnson & Johnson of New Jersey; although the trademark was originally applied to a felted absorbent cotton, more recently the trademark has been applied to synthetic material, such as rayon, that absorbs moisture) and the like. In most uses, the upper layer 102 is preferably the layer in contact with tissue, while the lower layer 104 provides wicking of biological fluids, such as blood, mucous, and other fluids encountered during the course of the surgical procedure. Some or all portions of the upper layer 102 can also be provided with a biological adhesive layer for securing the protective tissue cover 100 as needed. To prevent undue abrasion, the adhesive portions should be applied to areas where irritation from abrasion is minimal or non-existent. The flexibility of the upper layer 102 eases application of the protective tissue cover 100 onto tissue by easily conforming to the contours thereof. In addition, the protective tissue cover 100 can be provided with at least one perforation 106 so that the user can tear off smaller sections of the protective tissue cover 100, as desired.

As for the material of the lower layer 104 or the absorbent layer, it is preferable that this layer be made of material resistant to leaving residue. For example, many barriers exist that are constructed from cotton products. However, they tend to adhere to tissue or left in-situ unintentionally. This type of situation often results in unintended trauma to the tissue due to a peeling effect on the tissue when attempting to remove the same. Thus, while cotton products exhibit great absorption, they and similar products should be constructed to minimize adherence characteristics.

In this embodiment, the upper layer 102 can also be provided with at least one detection strip 108 embedded in the upper layer 102. As previously mentioned, the use and number of surgical equipment is closely monitored during surgery. There have been cases where cottonoid paddies or other implements have been accidentally overlooked and left in the body of the patient. This requires an immediate response and removal of the foreign matter before complications can occur. In order to avoid and minimize the dangers of such occurrences, especially from human error, the detection strip 108 permits early detection, via intra-operative or post-operative X-ray, CAT or MRI scan, of such potentially overlooked foreign matter. The composition of the detection strip 108 can include barium and compounds thereof, non-ferromagnetic metals, plastic or any other material that can provide discernible contrast in X-ray scans or molecular excitations. Other detection methods and materials can include coatings detectable via various bands of light.

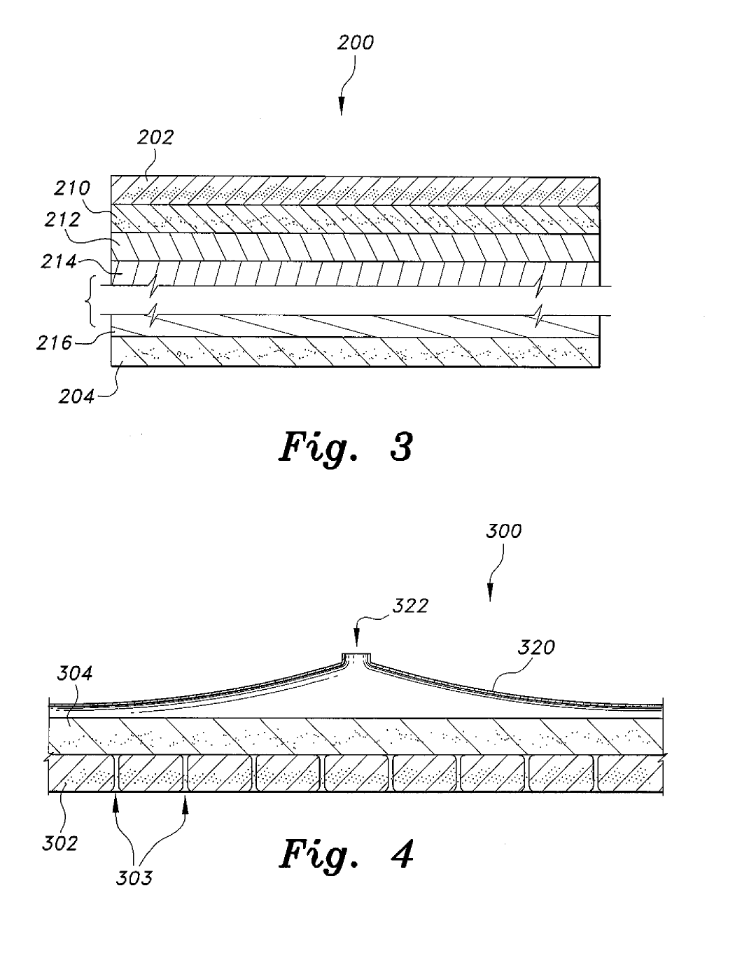

A still further alternative protective tissue cover 200 is shown in FIG. 3. In this exemplary embodiment, the protective tissue cover 200 is constructed from a plurality of plies of material 202, 204, 210, 212, 214, 216. As shown, the protective tissue cover 200 includes a first or upper layer 202 constructed from the same sterile and/or antimicrobial material mentioned above while the second or lower layer 204 is preferably constructed from absorbent material such as cottonoid and the like. The intermediate layers 210, 212, 214, 216 each can be composed of various materials tailored to the requirements of the user. For example, one of the intermediate layers can be a detection layer similar to the detection layer 108 mentioned above, another can be composed of controllable thermogenic materials that can retain cold or heat for a given or user-defined duration, and still another can be a medicament, with or without time-release properties. The multiple layered construction of the protective tissue cover 200 allows for a variety of ply configurations tailored for the needs of the surgical procedure and/or patient requirements.

A further alternative protective tissue cover 300 is shown in FIG. 4. In this embodiment, the protective tissue cover 300 includes features for easy removal of fluids during surgical procedures. As shown, the protective tissue cover 300 includes a first layer 302 and a second layer 304. The first layer 302 is constructed from the same sterile and/or antimicrobial material mentioned above, while the second 304 is preferably constructed from absorbent material, such as Cottonoid® and the like. Unlike the previous embodiments, the first layer 302 includes a plurality of microtubes 303 that permit flow of fluids between the layers. These microtubes 303 can be formed in various processes such as molding, laser or mechanical perforations, or chemically induced formations.

The protective tissue cover 300 can be provided with an outer shroud or cap 320 that surrounds select portions or all of the layers 302, 304. An adapter opening or nipple 322 is formed on the outer shroud 320 for selective attachment of a remote suction device (not shown). This configuration permits positive suctioning of excess fluids through the protective tissue cover 300 during the surgical procedure. As an alternative, the outer shroud 320 can be removed and suctioning can be performed on the exposed surfaces of the layers 302, 304.

It is to be understood that the present invention is not limited to the embodiments described above, but encompasses any and all embodiments within the scope of the following claims.

We claim:

1. A protective tissue cover, comprising an elongate, elastomeric sleeve having a closed end and an open end, the sleeve being adapted to be slidably attached to a working portion of a surgical retractor, the sleeve being constructed from smooth, sterile, antimicrobial material for minimizing potential contact irritation with tissue during a surgical procedure.

2. The protective tissue cover according claim 1, wherein said material is an elastomeric polymer impregnated with at least one antimicrobial selected from the group consisting of iodine, chlorhexadine, collagen, vitamin D, quaternary amines, halamine, and silver.

3. A protective tissue cover, comprising an elongate web having a plurality of layers, including at least one layer of smooth, sterile, antimicrobial material and at least one layer of absorbent material.

4. The protective tissue cover according to claim 3, wherein said at least one layer of smooth, sterile, antimicrobial material is a polymer impregnated with at least one antimicrobial selected from the group consisting of iodine, chlorhexadine, collagen, vitamin D, quaternary amines, halamine, and silver.

5. The protective tissue cover according to claim 4, wherein said at least one layer of absorbent material is at least one material selected from the group consisting of polymers and residue-resistant cotton.

6. The protective tissue cover according to claim 5, wherein said plurality of layers comprises an upper layer and a lower layer, said at least one layer of smooth, sterile and/or antimicrobial material being the upper layer and said at least one layer of absorbent material being the lower layer.

7. The protective tissue cover according to claim 6, further comprising a detection strip embedded in the upper layer.

8. The protective tissue cover according to claim 7, wherein said detection strip comprises at least one material selected from the group consisting of barium and salts thereof, non-ferromagnetic metals, plastic, and detectable coatings.

9. The protective tissue cover according to claim 6, wherein said plurality of layers further comprises at least one intermediate layer disposed between said upper layer and said lower layer.

10. The protective tissue cover according to claim 9, wherein said at least one intermediate layer comprises at least one material selected from the group consisting of a machine-scan detectable material, a controllable thermogenic material, and medicinal material.

11. The protective tissue cover according to claim 6, further comprising perforations extending across said web for tearing select desired lengths of the protective tissue cover.

12. The protective tissue cover according to claim 6, further comprising a plurality of microtubes formed through said upper layer, the microtubes permitting passage of fluid between said upper and lower layers.

13. The protective tissue cover according to claim 12, further comprising a suction shroud covering at least a portion of said upper layer, the suction shroud having an adapter for selective connection to a suction device.
